(19) United States
(12) Patent Application Publication
(10) Pub. No.: US 2008/0071132 A1
(43) Pub. Date: Mar. 20, 2008

(54) Brachytherapy apparatus and method for treating tissue forming an internal body cavity

(76) Inventors: Gary A. Lamoureux, Woodbury, CT (US); David Hart, Gilbert, AZ (US); Richard A. Terwilliger, Livingston, TX (US)

Correspondence Address: MCCARTER & ENGLISH LLP;CITYPLACE I, 185 ASYLUM STREET, HARTFORD, CT, 06103, US

(21) Appl. No.: 11/880,178

(22) Filed: Jul. 20, 2007

Related U.S. Application Data
(60) Provisional application No. 60/832,688, filed on Jul. 21, 2006.

(51) Int. Cl. A61M 36/12

(52) U.S. Cl. 600/7

(57) ABSTRACT

A brachytherapy apparatus and method are provided for treating tissue forming an internal body cavity, such as an excised breast or brain tumor bed. The apparatus has at least one outer wall that is movable between (i) a retracted position spaced inwardly relative to tissue forming the internal body cavity, and (ii) an expanded position spaced outwardly and contiguous to tissue forming the internal body cavity. The outer wall is formed by a stent, a balloon catheter, or a plurality of axially-elongated solid or hollow flexible members that are angularly spaced relative to each other. A plurality of radiation sources are carried by the at least one outer wall and movable therewith between the retracted and expanded positions for transmitting radiation in the expanded position into the tissue forming the internal body cavity.

### CROSS REFERENCE TO RELATED APPLICATIONS

This application claims priority to U.S. Provisional Application No. 60/832,688, filed Jul. 21, 2006, the contents of which are hereby incorporated by reference in their entirely as part of the present disclosure.

### FIELD OF THE INVENTION

The present invention relates to apparatus and methods for treating proliferative tissue disorders, and more particularly, to apparatus and methods for the treatment of such disorders by brachytherapy, such as breast brachytherapy.

### BACKGROUND INFORMATION

Malignant tumors can be treated by surgical resection of the tumor to remove as much of the tumor as possible. However, infiltration of the tumor cells into normal tissue surrounding the tumor can limit the therapeutic value of surgical resection because the infiltration can be difficult or impossible to treat surgically. Radiation therapy can be used to supplement surgical resection by targeting the residual malignant cells after resection, with the goal of sterilizing them, reducing the rate of recurrence or delaying the time to recurrence. Radiation therapy can be administered through one of several methods, or a combination of methods, including brachytherapy and external-beam radiation.

Brachytherapy refers to radiation therapy delivered by a spatially confined source of therapeutic rays (such as radioactive seeds) inserted into the body at or near a tumor or other proliferative tissue disease site. For example, brachytherapy can be performed by implanting radiation sources directly into the tissue to be treated. In brachytherapy, radiation doses are highest in close proximity to the radiotherapeutic source, providing a high tumor dose while sparing surrounding normal tissue. Brachytherapy is useful for treating malignant brain and breast tumors, among others.

One of the drawbacks associated with certain prior art breast brachytherapy apparatus and methods is that a single source of radiation is inserted into a central region of the resected tumor cavity. As a result, it can be difficult to apply a substantially uniform radiation intensity to the walls of the tumor bed. Alternatively, it can be difficult to selectively apply different levels of radiation intensity to different regions of the resected tumor bed.

It is an object of the present invention to overcome one or more of the above-described drawbacks and/or disadvantages of the prior art.

### SUMMARY OF THE INVENTION

In accordance with a first aspect, the present invention is directed to a brachytherapy apparatus for treating tissue forming an internal body cavity, such as an excised breast or brain tumor bed. The apparatus comprises at least one outer wall movable between (i) a retracted position spaced inwardly relative to tissue forming the internal body cavity, and (ii) an expanded position spaced outwardly and contiguous to tissue forming the internal body cavity. A plurality of radiation sources of the apparatus are coupled to the at least one outer wall, and are movable therewith between the retracted and expanded positions for transmitting radiation in the expanded position into the tissue forming the internal body cavity.

In some embodiments of the present invention, the apparatus defines an elongated axis, and the at least one outer wall is movable radially relative to the axis between the retracted and expanded positions. The plurality of radiation sources are spaced relative to each other, and can be embedded in, can form, or otherwise can be mounted on, the at least one outer wall of the apparatus. Preferably, in the expanded position, the plurality of radiation sources are substantially uniformly radially spaced relative to the respective contiguous tissue surface portions forming the internal body cavity.

In one such embodiment of the present invention, the at least one outer wall is defined by a plurality of axially-elongated, radiation-emitting members angularly spaced relative to each other and movable between (i) the retracted position spaced radially inwardly toward the elongated axis, and (ii) the expanded position spaced radially outwardly relative to the elongated axis. Each of a plurality of the radiation-emitting members includes at least one radiation source for transmitting radiation into the adjacent tissue forming the internal body cavity. Preferably, in the expanded position, the at least one outer wall and/or tissue forming the internal body cavity substantially conformably contacts the other. In some embodiments of the present invention, the at least one outer wall is flexible and substantially conforms to the tissue forming the internal body cavity. If desired, the flexible radiation-emitting members can be defined by strands or like flexible structures, wherein each strand carries one or more radiation sources, such as radioactive wires or seeds. In one such embodiment, the radiation-emitting members are defined by one or more first elongated half-shall members, and one or more corresponding second elongated half-shell members fixedly secured to the first elongated half-shell members with the radiation sources located therebetween. In one embodiment, a plurality of the radiation-emitting members define a plurality of axially-elongated apertures therebetween. In one such embodiment, a plurality of the radiation-emitting members are interconnected by a plurality of webs extending therebetween. In one such embodiment, a plurality of the webs define a plurality of axially-extending apertures formed between adjacent radiation-emitting members.

In some embodiments of the present invention, the outer wall is defined by a flexible membrane extending about a periphery of the apparatus. In one such embodiment, the flexible membrane is defined by a balloon, such as a balloon catheter or balloon-tipped catheter. In one such embodiment, the apparatus comprises an external flexible membrane, and an internal flexible membrane spaced radially inwardly relative to the external flexible membrane. The plurality of radiation sources are located between the internal and external membranes. In one such embodiment, the apparatus further comprises a plurality of axially-elongated, radiation-emitting members that are angularly spaced relative to each other, that include thereon the plurality of radiation sources, and that are movable with the external flexible membrane between the retracted and expanded positions.

In another embodiment of the present invention, the at least one outer wall is defined by a stent. In one such embodiment, the stent includes on an outer region thereof a plurality of strands or like flexible, axially-extending, radiation-emitting members. In some embodiments of the present invention, each radiation source is a low dose radiation source, and each low dose radiation source is selected from the group including a radioactive wire and a radioactive seed. In one such embodiment, each of a plurality of the radiation-emitting members is defined by a flexible strand including therein a plurality of radioactive seeds. In one such embodiment, each strand is formed of a material that is biocompatible and biodegradable.

In one embodiment of the present invention, at least one radiation source defines a first predetermined radiation intensity, at least one second radiation source defines at least one second predetermined radiation intensity greater than the first predetermined radiation intensity, and the first and second radiation sources are spaced relative to each other at prescribed locations on the at least one outer wall. The apparatus further comprises indicia representing the location of the first and/or second radiation sources to facilitate orienting the apparatus within the internal body cavity.

In one embodiment of the present invention, the apparatus further comprises a first support member and a second support member. The at least one outer wall is coupled to the first and/or second support members, and at least one of the first and second support members is movable relative to the other to, in turn, move the outer wall between the retracted position and the expanded position. In one such embodiment, the first support member is substantially coaxially mounted within the second support member. In one such embodiment, the first support member includes a proximal end and a distal end, and the distal end includes thereon a stop surface that engages the second member to facilitate moving the outer wall between the retracted position and the expanded position.

In accordance with another aspect, the present invention is directed to brachytherapy apparatus for treating tissue forming an internal body cavity. The apparatus comprises first means movable between (i) a retracted position spaced inwardly relative to tissue forming the internal body cavity, and (ii) an expanded position spaced outwardly and contiguous to tissue forming the internal body cavity. The apparatus further comprises second means movable with the first means between the retracted and expanded positions for transmitting radiation in the expanded position into the tissue forming the internal body cavity. In some embodiments of the apparatus, the first means is defined by an outer wall of the apparatus, and the second means is defined by a plurality of radiation sources. In some such embodiments, the outer wall is defined by (i) a stent, (ii) a balloon catheter or balloon-tipped catheter, (iii) a plurality of radiation-emitting members angularly spaced relative to each other and movable radially between the retracted position and the expanded position, or (iv) a plurality of flexible strands each including therein at least one radioactive seed.

In accordance with another aspect, the present invention is directed to a method for treating tissue forming an internal body cavity, such as an excised breast or brain tumor bed. The method comprises the following steps:

(i) resecting a tumor site to remove at least a portion of a cancerous tumor and, in turn, forming an internal body cavity at the tumor site;

(ii) providing a brachytherapy apparatus including at least one outer wall movable between a retracted position and an expanded position, and a plurality of radiation sources movable with the at least one outer wall between the retracted position and the expanded position;

(iii) inserting the brachytherapy apparatus with the at least one outer wall in the retracted position into the internal body cavity at the tumor site;

(iv) moving the at least one outer wall and radiation sources from the retracted position to the expanded position; and

(v) transmitting radiation from the plurality of radiation sources into the tissue forming the internal body cavity at the tumor site.

In some embodiments of the present invention, the method further comprises the step of moving the at least one outer wall into contact with the tissue forming the internal body cavity at the tumor site. In some such embodiments, the method further comprises the step of substantially conforming the shape of the at least one outer wall in the expanded position and/or the shape of the internal body cavity to the shape of the other.

In some embodiments of the present invention, the method further comprises the step of inflating the apparatus with a fluid to move the at least one outer wall from the retracted position to the expanded position. In other embodiments of the present invention, the method further comprises the step of manually manipulating the apparatus and mechanically moving the at least one outer wall from the retracted position to the expanded position.

Some embodiments of the present invention further comprise the step of substantially uniformly spacing each radiation source relative to the contiguous surface portion of tissue forming the internal body cavity.

In some embodiments of the present invention, the method further comprises the step of providing a brachytherapy apparatus including a plurality of low dose radiation sources, and maintaining the apparatus in the expanded position within the internal body cavity for a substantial treatment period, e.g., greater than about five hours.

In some embodiments of the present invention, the method further comprises the steps of providing a brachytherapy apparatus wherein the at least one outer wall is defined by a plurality of axially-elongated, flexible, tubular members angularly spaced relative to each other, and moving the plurality of tubular members from the retracted position to the expanded position into contact with the tissue forming the internal body cavity. In some embodiments of the present invention, the method comprises the step of filling a plurality of tubular members, each with at least one low dose radiation source. The tubular members can be filled with radiation sources in accordance with a pre-planned method wherein the tubular members are filled with the radiation sources at the factory or otherwise prior to unpackaging the device in the operating room. This method also may be referred to as a “pre-configured” or “pre-loaded” method and may be employed in situations wherein the practitioner knows the desired radiation dosage cloud for a tumor bed or site prior to removing the tumor such that a “standardized” or factory loaded brachytherapy apparatus may be employed. Alternatively, the tubular members can be filled with radiation sources in accordance with an intraoperative method wherein the tubes are loaded with the radiation sources in the operating room, or at about the time of the operation, in accordance with a physician's prescription for the respective tumor site or bed.

In other embodiments of the present invention, the method comprises the steps of (a) connecting at least a plurality of the tubular members in the expanded position to a source of high dose radiation; (b) introducing the source of high dose radiation into the plurality of tubular members; and (c) emitting the high dose radiation into the tissue forming the internal body cavity. Preferably, the method further comprises the steps of (d) emitting the high dose radiation for a prescribed treatment period; (e) after expiration of the prescribed treatment period disconnecting the plurality of tubular members from the source of high dose radiation; (f) maintaining the brachytherapy apparatus within the internal body cavity throughout a prescribed non-treatment period; and (g) after expiration of the prescribed non-treatment period, repeating at least once steps (a) through (e).

In one embodiment of the present invention, the method further comprises the step of providing a brachytherapy apparatus including at least one radiation source defining a first predetermined radiation intensity, and at least one second radiation source defining a second predetermined radiation intensity greater than the first predetermined radiation intensity. The method further comprises the step of positioning the brachytherapy apparatus within the internal body cavity such that the first radiation source is contiguous to a first tissue region prescribed to receive a relatively lower dose of radiation, and the second radiation source is contiguous to a second tissue region prescribed to receive a relatively higher dose of radiation in comparison to the first tissue region.

One advantage of the apparatus and method of the present invention is that the at least one outer wall or like means is movable between the retracted and expanded positions, and the plurality of radiation sources are coupled thereto and movable therewith. As a result, the radiation sources, such as low dose radioactive seeds or wires, or high dose radiation sources, can be positioned contiguous to the target tissue, and if desired, the plural radiation sources can be substantially uniformly spaced relative to the respective target tissue.

Other objects and advantages of the present invention will become more readily apparent in view of the following detailed description of the preferred embodiments and accompanying drawings.

### BRIEF DESCRIPTION OF THE DRAWINGS

FIG. 1A is a somewhat schematic, side elevational view of a first embodiment of a brachytherapy apparatus of the present invention showing the apparatus in a retracted position for insertion into an internal body cavity, such as an excised breast or brain tumor bed.

FIG. 1B is a somewhat schematic, side elevational view of the brachytherapy apparatus of FIG. 1A in the expanded position for contacting the tissue wall of the internal body cavity and emitting prescribed dosages of radiation thereto.

FIG. 2A is a top elevational view of a mold for forming a web of radiation-emitting members forming the expansible outer wall of the brachytherapy apparatus of FIGS. 1A and 1B, or the web formed in such a mold.

FIG. 2B is somewhat schematic, perspective view of the expansible outer wall of FIG. 2A shown after it is cut to a desired length and formed into a tubular structure.

FIG. 2C is a somewhat schematic, perspective view of the outer wall of FIG. 2B shown with the axially-elongated apertures formed between the flexible radiation-emitting members.

FIG. 3 is a schematic illustration in accordance with another embodiment of the present invention of plural radiation sources of different predetermined intensities located in predetermined positions on the brachytherapy apparatus to selectively treat different tissue regions of an internal body cavity with different prescribed radiation dosages.

FIGS. 4A and 4B are somewhat schematic views of another embodiment of the present invention including a stent for moving the outer wall and radiation sources between the retracted position, as shown in FIG. 4A, and the expanded position, as shown in FIG. 4B.

FIG. 5A is a somewhat schematic view of another embodiment of the present invention wherein the outer wall carrying the radiation sources is formed by a balloon catheter, and FIG. 5B is a somewhat schematic, cross-sectional view of the apparatus of FIG. 5A showing internal and external flexible membranes with the radiation sources located therebetween.

### DETAILED DESCRIPTION OF PREFERRED EMBODIMENTS

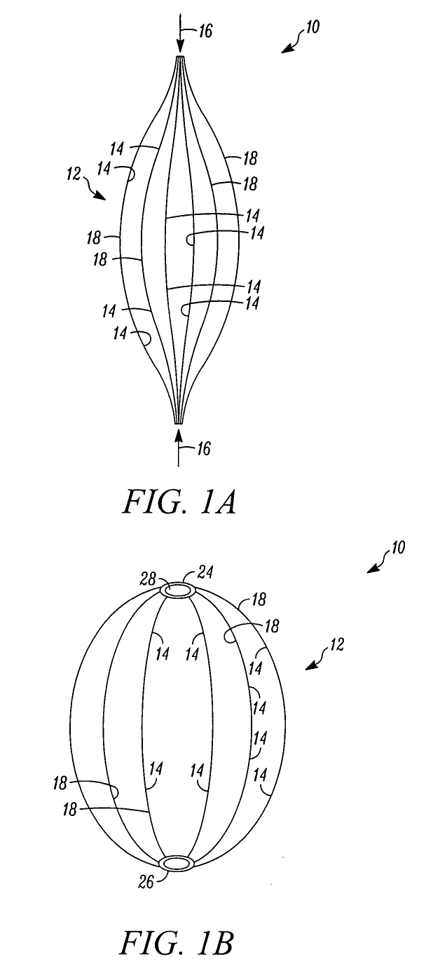

In FIGS. 1A and 1B an apparatus embodying the present invention is indicated generally by the reference numeral 10. The apparatus 10 comprises an outer wall 12 movable between (i) a retracted position, as shown in FIG. 1A, spaced inwardly relative to tissue forming an internal body cavity, such as an excised breast or brain tumor bread, and (ii) an expanded position, as shown in FIG. 1B, spaced outwardly and contiguous to tissue forming the internal body cavity. A plurality of radiation sources indicated typically at 14 are coupled to the outer wall 12, and are movable therewith between the retracted and expanded positions for transmitting radiation in the expanded position into the tissue forming the internal body cavity.

In the illustrated embodiment, the apparatus 10 defines an elongated axis 16, and the outer wall 12 is movable radially relative to the axis between the retracted and expanded positions. The plurality of radiation sources 14 are spaced relative to each other, and are embedded in, or otherwise mounted on, the outer wall 12. If desired, the radiation sources 14 may form the outer wall, such as wherein the radiation sources 14 take the form of flexible radioactive wires or strands. In the illustrated embodiment, in the expanded position, the plurality of radiation sources 14 are substantially uniformly radially spaced relative to the respective contiguous tissue surface portions forming the internal body cavity.

Also in the illustrated embodiment, the outer wall 12 is defined by a plurality of axially-elongated, radiation-emitting members 18 that are angularly spaced relative to each other and movable between (i) the retracted position, as shown typically in FIG. 1A, spaced radially inwardly toward the elongated axis 16, and (ii) the expanded position, as shown typically in FIG. 1B, spaced radially outwardly relative to the elongated axis 16. In the illustrated embodiment, each radiation-emitting member 18 includes at least one, and preferably several radiation sources 14 for transmitting radiation into the adjacent tissue forming the internal body cavity. The radiation sources may be loaded at any time, and in accordance with any of numerous different loading methods that are currently known, or that later become known, such as by pre-loading or intraoperative loading. As described further below, the radiation sources 14 may take the form of any of numerous different radiation sources that may be prescribed in a treatment plan, or otherwise that are currently known, or that later become known, for brachytherapy or otherwise for use in connection with the apparatus of the present invention. Further, the radiation sources 14 may be applied to the outer wall(s) of the apparatus in any of numerous different configurations or patterns that are currently known, or that later become known. Still further, and as described further below, the apparatus 10 may comprise different types of radiation sources 14, and/or radiation sources 14 of different intensities in order to provide a prescribed radiation treatment or otherwise as desired.

In the illustrated embodiment, in the expanded position, as shown typically in FIG. 1B, the outer wall 12 and tissue forming the internal body cavity substantially conformably contact each other. Preferably, the radiation-emitting members 18 are sufficiently flexible to substantially conformably contact the contiguous tissue forming the internal body cavity. However, if desired, the tissue forming the internal body cavity may conform in part to the shape of one or more of the radiation-emitting members 18. In the illustrated embodiment, the flexible radiation-emitting members 18 are defined by strands or like flexible structures, wherein each strand carries at least one, and preferably a plurality of radiation sources axially spaced relative to each other. In one embodiment of the present invention, the radiation sources 14 are radioactive seeds. In another embodiment of the present invention, the radiation sources 14 are radioactive wires.

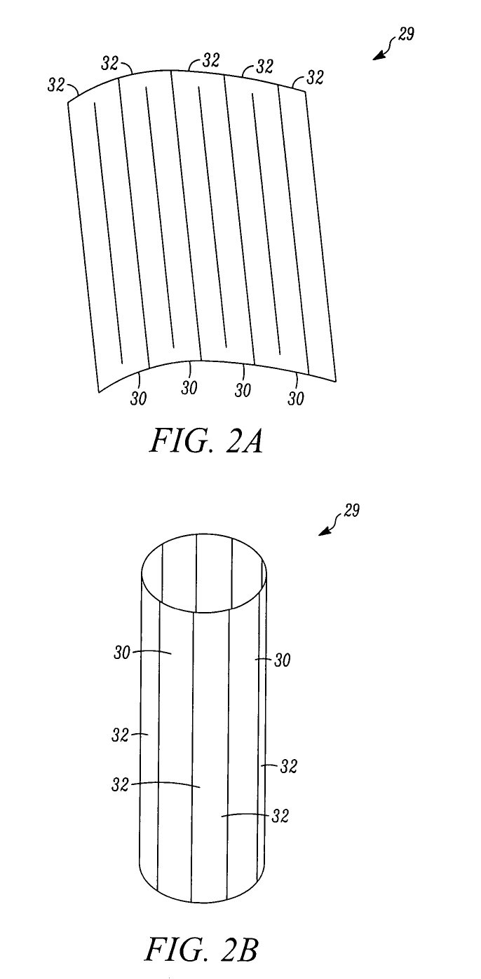

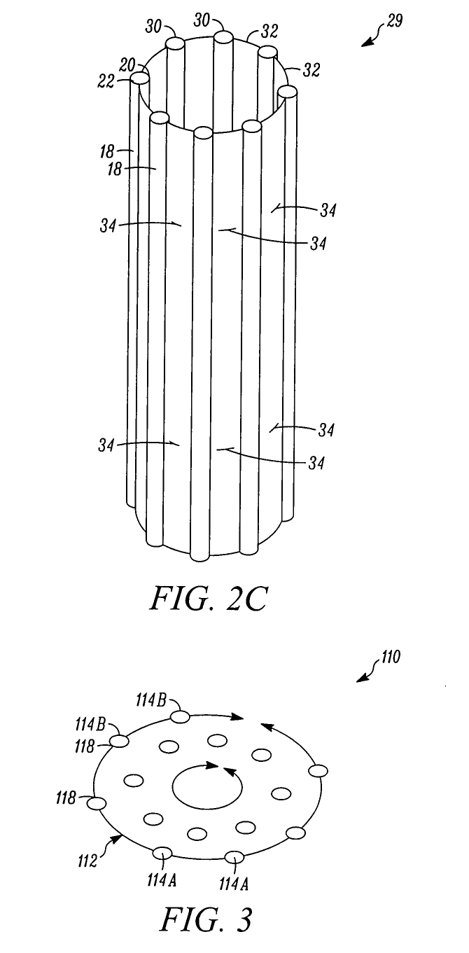

In one embodiment of the present invention, and as shown typically in FIGS. 2B-2C, the radiation-emitting members forming the at least one outer wall 12 are defined by a plurality of axially-elongated, flexible, tubular members 18 angularly spaced relative to each other. The tubular members 18 may receive any of numerous different radiation sources that are currently known, or that later become known, such as radioactive seeds or wires. As may be recognized by those of ordinary skill in the pertinent art based on the teachings herein, the usage of one or more radioactive wires may facilitate creating a more homogenous dosage cloud in comparison to the usage of spaced seeds. As shown typically in FIG. 2A, the apparatus 10 can be made by forming a sheet-like structure 29 defining a plurality of axially-elongated, substantially parallel tubular portions 30 laterally spaced relative to each other, and a plurality of web portions 32 formed therebetween. The sheet-like structure 29 may be formed in any of numerous different ways that are currently known, or that later become known, such as by any of numerous different extrusion and/or molding processes. If desired, the tubular members 18 may be individually molded, extruded or otherwise formed. If desired, the tubular members 18 can be attached to a web or sheet-like substrate, or can be attached at distal and proximal ends thereof to a coaxially located support device for insertion through a catheter or otherwise into a resected tumor bed or site in a contracted position, and for moving the outer wall(s) into an expanded position within the bed or site. Alternatively, and as shown typically in FIG. 2C, the sheet-like structure 29 may be defined by one or more first elongated half-shall members 20, and one or more corresponding second elongated half-shell members 22 fixedly secured to the first elongated half-shell members. If desired, one or more of the individual tubular portions 30 may be formed of first and second elongated half-shell members. In addition, the sheet-like structure 29 may be formed of different materials. For example, the tubular portions 30 may be formed of a first material, and the web portions 32 may be formed of a second material. In one such embodiment, the web portions 32 are co-molded with the tubular portions 32. In addition, the tubular and web portions 30 and 32, respectively, may be formed of any of numerous different materials that are currently known, or that later become known for performing the function of the invention as disclosed herein. For example, the tubular portions 30 and/or web portions 32 may be formed of materials that are biocompatible, and if desired, biodegradable.

As shown typically in FIG. 2B, the sheet-like structure 29 is cut or otherwise formed to the desired length, and is then rolled or folded on itself to form a tubular structure, defining a desired tubular diameter. The adjacent web portions 32 formed at the ends of the sheet-like structure 29 are fixedly secured to each other, such as by using an adhesive, ultrasonic welding, or any of numerous other processes or mechanisms for fixedly securing the ends together to form the tubular structure, that are currently known, or that later become known. As shown in FIG. 2C, slits or axially-elongated apertures 34 are formed in the web portions 32 between adjacent tubular portions 30 to facilitate or otherwise allow the tubular portions 30 forming the radiation-emitting members 18 to flex or bow outwardly from the retracted position, as shown typically in FIG. 1A, to the expanded position, as shown typically in FIG. 1B.

As shown schematically in FIG. 1B, the distal end of the tubular structure of FIG. 2C is mounted to a first support member 24, and the proximal end of the tubular structure is mounted to a second support member 26. The tubular structure 29 of FIG. 2C forming the outer wall 12 is connected to the first and second support members 24 and 26, respectively, and the first and second support members are movable relative to each other to, in turn, move or bow the outer wall 12 between the retracted position, as shown typically in FIG. 1A, and the expanded position, as shown typically in FIG. 1B. In the illustrated embodiment, the first support member 24 is substantially coaxially mounted within the second support member 26. The first support member 24 includes a proximal end (not shown) and a distal end 28, and the distal end defines a stop surface that engages the distal end of the outer wall 12 (or tubular structure 29) to facilitate moving or bowing the radiation-emitting members 18 between the retracted position of FIG. 1A and the expanded position of FIG. 1B.

In the operation of the apparatus 10, the apparatus is used to treat tissue forming an internal body cavity, such as an excised breast or brain tumor bed. A method of using the apparatus 10 comprises the following steps:

(i) resecting a tumor site to remove at least a portion of a cancerous tumor and, in turn, forming an internal body cavity at the tumor site;

(ii) inserting the apparatus 10 with the outer wall 12 in the retracted position of FIG. 1A into the internal body cavity at the tumor site;

(iii) moving the outer wall 12 and radiation sources 14 thereon from the retracted position of FIG. 1A to the expanded position of FIG. 1B; and

(iv) transmitting radiation from the plurality of radiation sources 14 into the tissue forming the internal body cavity at the tumor site.

In the preferred embodiments of the present invention, the outer wall 12 is moved into contact with the tissue forming the internal body cavity at the tumor site. Preferably, the method further comprises the step of substantially conforming the shape of the outer wall 12 in the expanded position to the shape of the internal body cavity, and/or substantially conforming the shape of the internal body cavity to the shape of the outer wall 12. In some embodiments of the present invention, the method further comprises the step of substantially uniformly spacing each radiation source 14 relative to the contiguous surface portion of tissue forming the internal body cavity.

In some embodiments of the present invention, the apparatus 10 may include a plurality of low dose radiation sources, and the apparatus is maintained in the expanded position within the internal body cavity for a substantial treatment period, e.g., greater than about five hours, such as several days. Alternatively, the apparatus 10 may be used with high dose radiation sources. In such embodiments, the apparatus 10 is used by (a) connecting the tubular radiation-emitting members 18 in the expanded position to a source of high dose radiation, such as an after loader of a type known to those of ordinary skill in the pertinent art containing a source of high dose radiation; (b) introducing the high dose radiation into the tubular members 18; and (c) emitting the high dose radiation into the tissue forming the internal body cavity. In these embodiments, the method further comprises the steps of (d) emitting the high dose radiation for a prescribed treatment period; (e) after expiration of the prescribed treatment period disconnecting the tubular members 18 from the source of high dose radiation; (f) maintaining the apparatus 10 within the internal body cavity throughout a prescribed non-treatment period; and (g) after expiration of the prescribed non-treatment period, repeating at least once steps (a) through (e).

In FIG. 3, another apparatus embodying the present invention is indicated generally by the reference number 110. The apparatus 110 is substantially similar to the apparatus 10 described above, and therefore like reference numerals preceded by the numeral “1” are used to indicate like elements. A primary difference of the apparatus 110 in comparison to the apparatus 10 above is that the apparatus 110 includes one or more first radiation sources 114A defining a first predetermined radiation intensity, and one or more second radiation sources 114B defining a second predetermined radiation intensity greater than the first predetermined radiation intensity. In use, the brachytherapy apparatus 110 is positioned within the internal body cavity such that the first radiation source(s) 114A is contiguous to a first tissue region prescribed to receive a relatively lower dose of radiation, and the second radiation source(s) 114B is contiguous to a second tissue region prescribed to receive a relatively higher dose of radiation in comparison to the first tissue region. For example, the lower dose radiation source(s) 114A may be located adjacent to a thinner tissue region than the higher dose radiation source(s) 114B. Preferably, the apparatus 110 further comprises indicia (not shown) representing the location of the first and/or second radiation sources 114A, 114B to facilitate orienting the apparatus within the internal body cavity in accordance with a prescribed radiation treatment. As may be recognized by those of ordinary skill in the pertinent art based on the teachings herein, the apparatus 110 may include any desired number of first and second radiation sources, each radiation source may define any desired radiation intensity, the apparatus may include radiation sources having any desired number of different radiation intensities, and the plural radiation sources may define any desired radiation intensity pattern.

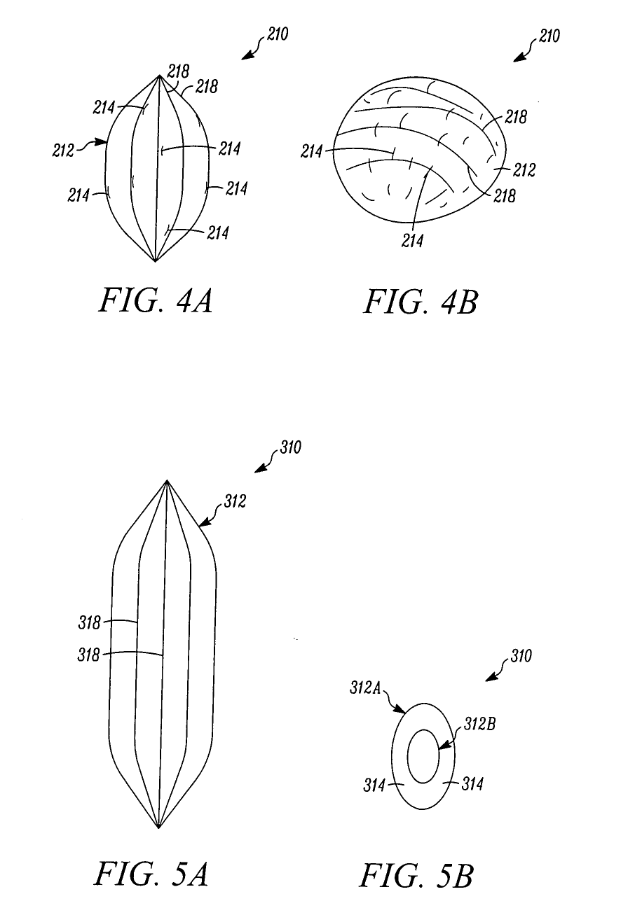

In FIGS. 4A and 4B another apparatus embodying the present invention is indicated generally by the reference numeral 210. The apparatus 210 is substantially similar to the apparatus 10 and 110 described above, and therefore like reference numerals preceded by the numeral “2”, or preceded by the numeral “2” instead of the numeral “1”, are used to indicate like elements. A primary difference of the apparatus 210 in comparison to the apparatus 10 and 110 described above, is that the outer wall 212 is defined by a stent that includes on an outer region thereof a plurality of strands or like flexible, axially-extending, radiation-emitting members 218. As shown in FIGS. 4A and 4B, the stent 212 is movable between a retracted position as shown in the upper portion of the drawing, to an expanded position as shown in the lower portion of the drawing such that the radiation-emitting members 218 and radiation sources 214 thereon are moved into contact with tissue forming the internal body cavity of, for example, an excised breast or brain tumor site. In some embodiments of the apparatus 210, each radiation source 214 is a low dose radiation source, and each low dose radiation source is selected from the group including a radioactive wire and a radioactive seed. In one such embodiment, each of a plurality of the radiation-emitting members 214 is defined by a flexible strand or tube including therein a plurality of radioactive seeds which can be preloaded or intraoperatively loaded.

As may be recognized by those of ordinary skill in the pertinent art based on the teachings herein, the stent may take the form of any of numerous different stents that are currently known, or that later become known. For example, the stent may take the form of a woven device that expands when co-axial pressure is applied thereto, or that is spring loaded such that a spring force causes the outer wall(s) of the stent to move from a contracted position to an expanded position. In addition, the stent may include any of numerous different types of radioactive sources that are currently known, or that later become known. For example, if desired, the outer walls of the stent may be defined by a plurality of radioactive wires or other materials that emit the desired dosage of radiation and that are expandable between contracted and expanded positions.

In FIGS. 5A and 5B another apparatus embodying the present invention is indicated generally by the reference numeral 310. The apparatus 310 is substantially similar to the apparatus 10, 110 and 210 described above, and therefore like reference numerals preceded by the numeral “3”, or preceded by the numeral “3” instead of the numerals “1” or “2”, are used to indicate like elements. A primary difference of the apparatus 310 is that the outer wall 312 is defined by a flexible membrane extending about a periphery of the apparatus. Preferably, the flexible membrane is defined by a balloon, such as a balloon catheter or balloon-tipped catheter. In one such embodiment, the apparatus 310 comprises an external flexible membrane 312A, and an internal flexible membrane 312B spaced radially inwardly relative to the external flexible membrane. The plurality of radiation sources 314 are located between the internal and external membranes 312B and 312A, respectively. In one such embodiment, a tubular structure 29 of the type shown in FIG. 2C is located between the internal and external flexible membranes 312B and 312A, respectively. In this embodiment, the tubular structure 29 defines a plurality of axially-elongated, radiation-emitting members 318 that are angularly spaced relative to each other, that include thereon the plurality of radiation sources 314, and that are movable with the external flexible membrane 312A between the retracted and expanded positions. The flexible membranes 312A and 312B can be constructed to move between the retracted and expanded positions in any of numerous different ways that balloon catheters are currently manipulated, or in the future are manipulated between retracted and expanded positions, such as by introducing a fluid (air or saline, for example) into the interior of the internal flexible membrane.

As may be recognized by those of ordinary skill in the pertinent art based on the teachings herein, the radiation-emitting members, the at least one outer wall, and/or the radiation sources of the apparatus, may take the form of any of numerous different such components or features that are currently known, or that later become known. For example, each radiation-emitting member 18, 118, 218 and 318 may take the form of a substantially axially, semi-rigid and radially or laterally flexible elongated member made of material which is bio-absorbable in living tissue. A plurality of radioactive seeds 14, 114, 214 and 314 may be encapsulated and positioned in a predetermined array in each respective radiation-emitting member in a predetermined and desired spaced relationship. The seeds can be of various types, including without limitation those having low energy and low half-life that are currently known, or that later become known. Exemplary seeds include Iodine seeds, such as I-125 seeds, which can be made using a welded titanium capsule containing iodine 125 adsorbed on a silver rod, Palladium 103 seeds, and Cesium 131 seeds. Additional examples of radioactive seeds that can be used in the radiation-emitting members are disclosed in U.S. Published Patent Application No. 2004/0102671 A1 and other patents and patent applications incorporated by reference below. The substantially axially, semi-rigid, and radially flexible elongated members 18, 118, 218 and 318 may be made of any of numerous different natural and/or synthetic bio-compatible and bio-absorbable materials, such as any of numerous different natural and synthetic polymers and copolymers, that are currently known or that later become known. Exemplary such materials are disclosed in U.S. Published Patent Application No. 2004/0102671 A1 and other patents and patent applications incorporated by reference below.

The radiation-emitting members 18, 118, 218 and 318 may be fashioned with any of numerous different manufacturing methods that are currently known, or that later become known, such as insert molding or compression molding. The radioactive seeds 14, 114, 214 and 314 may be placed into a fixture that spaces the seeds at the appropriate intervals in a cavity that is shaped to the desired final dimensions of the radiation-emitting members in accordance with a respective treatment plan. All the spacings can be of different lengths, if the preoperative therapeutic plan so specifies. A synthetic polymer is introduced into the mold at a temperature that is above the melt point of the polymer. The polymer flows around the seeds within the cavity, surrounds the seeds and fills in the spaces between the seeds. After the mold has cooled, it is disassembled, and the finished radiation-emitting members are removed. If desired, the finished radiation-emitting members ejected from the mold may take the form of the sheet-like structure 29 described above.

Alternatively, the tubular portions 30, 130, 230 and 330 encapsulating the radioactive seeds 14, 114, 214 and 314 may be formed as solid tubular members using compression molding techniques. Compression molding forms the molded piece in a two part mold where a polymer material is placed within the cavities of the mold in a liquid state. The seeds 14, 114, 214 and 314 are placed in position within the cavities filled with the polymer and the mold is closed and compressed, then cooled to form a piece that conforms to the shape of the closed cavity. If desired, the molded piece may take the form of the sheet-like structure 29 described above.

In another alternative embodiment, the radiation-emitting members 18, 118, 218 and 318 can be fashioned from two or more half-shells made from the same material as indicated above. The radiation-emitting members or strands are fashioned by sealing the seed elements 14, 114, 214 and 314 between the two or more elongate half-shells and fusing the half-shells by heat or other desired method. The seed elements can be placed within the half-shells and liquid material or polymer can be flowed into the unassembled or assembled half-shells, filling substantially all spaces not occupied by seed elements or spacers. If desired, the fused half shells may take the form of the sheet-like structure 29 described above.

In another embodiment, the radiation-emitting members 18, 118, 218 and 318 can be fashioned by producing a catheter or hollow member by extrusion. The material or polymer is placed into a chamber having a die and a mandrel. Pressure is applied by a piston in order to push the material through an opening between the die and the mandrel. During this process, the opening of the die forms the template for the outer walls of the catheter while the mandrel forms the interior bores. The radioactive seeds may then be inserted into the bores of the catheter during the extrusion process or thereafter. If desired, the extruded part may take the form of the tubular structure illustrated above in FIG. 2C that could be pre-loaded or intraoperatively loaded. The radioactive seeds 14, 114, 214 and 314 may be spaced at variable intervals specific to the treatment goals of the end user, and/or radioactive wires can be inserted into the tubular members. All the spacings can be of different lengths, if the preoperative therapeutic plan so specifies.

If desired, the manufacturing process can make the radiation-emitting members 18, 118; 218 and 318 and/or other desired features of the apparatus echogenic. In the case of the molding of the radiation-emitting members, air can be entrapped in the polymer material. During the cooling stage of the molding process, the mold is placed in a vacuum chamber and the air in the chamber is evacuated. This causes the entrapped air in the mold to come out of solution from the polymer, and as the mold cools, this air is entrapped within the cooling polymer in the form of minute bubbles suspended in the plastic. Air is a strong reflector of ultrasound energy, since the inherent impedance of air is many times greater than body tissue. When the radiation-emitting members are introduced into the body and imaged with ultrasound, the radiation-emitting members are clearly visible in the resulting image, and are thus echogenic. Alternatively, echogenic spacing elements containing air pockets can be placed between the seeds to facilitate ultrasound imaging thereof.

As is known in the industry, there is software which can be used to provide brachytherapy treatment planning guides which are customized for each individual patent. An example of such software is provided by Rossmed which is located at Ross Medical, 7100 Columbia Gateway Drive, Suite 160, Columbia, Md. 21046. This particular software, which is incorporated herein by reference, is known as the Strata suite, which software helps physicians to develop and visualize low dose rate brachytherapy treatment plans for treating malignant tumors in human tissue. The treatments entail the use of radioactive seed sources which are implanted adjacent to the malignant tissue. The Strata software uses imaging to create a three dimensional reconstruction of the patient's anatomy. The software is able to plan the placement of the seeds within the target. The radiation dose that is delivered to the target can be computerized and visualized using the software. The software can then be used to identify an optimal number of radiation-emitting members along with optimal seed dosages and spaces between seeds.

As disclosed in U.S. Published Patent Application No. 2004/0102671 A1 incorporated by reference below, the software can be used to prepare a prescription which optimizes the number of strands or other radiation-emitting members, and placement and spacing of seeds for each of the strands or other radiation-emitting members. This optimization plan can then be sent to a manufacturing site. An optimized strand or other radiation-emitting member can be created with the specified number of seeds and the specified distances between each seed pair. Once this prescription is filled at the manufacturing site, the custom strand or other radiation-emitting member can be sent back to the physician for treatment of the patient. With such an arrangement, radiation patterns can be optimally established for the treatment of each patient. Further the preparation time for the physician is greatly diminished as the physician does not have to hand assemble and hand load the seeds and spacers into a needle. As may recognized by those of ordinary skill in the pertinent art based on the teachings herein, such software and techniques can be used, or modified if necessary, to optimally establish radiation patterns for the various embodiments of the present invention for optimal treatment of respective patients.

Accordingly, the radiation-emitting members of the apparatus of the present invention may take the form of, or may be manufactured in accordance with the teachings of any of the following patents and patent applications that are hereby incorporated by reference in their entireties as part of the present disclosure: U.S. patent application Ser. No. 10/035,083 entitled “Delivery System and Method for Interstitial Radiation Therapy,” by Terwilliger et al., filed Dec. 28, 2001 (Attorney Docket No. WORLD-01000US1); U.S. Provisional Patent Application No. 60/360,237 entitled “System for Manufacturing Interstitial Radiation Therapy Seed Strands,” by Terwilliger et al., filed Feb. 26, 2002 (Attorney Docket No. WORLD-01000US3); U.S. Provisional Patent Application No. 60/360,272 entitled “Delivery System and Method for Interstitial Radiation Therapy Using Strands Constructed With Extruded Strand Housing,” by Terwilliger et al., filed Feb. 26, 2002 (Attorney Docket No. WORLD-01000US4); U.S. patent application Ser. No. 10/162,546 entitled “System for Manufacturing Interstitial Radiation Therapy Seed Strands,” by Terwilliger et al., filed Jun. 4, 2002 (Attorney Docket No. WORLD-01000US6); U.S. patent application Ser. No. 10/162,006 entitled “Delivery System and Method for Interstitial Radiation Therapy Using Strands Constructed with Extruded Strand Housings,” by Terwilliger et al., filed Jun. 4, 2002 (Attorney Docket No. WORLD-01000US7); U.S. patent application Ser. No. 10/397,940 entitled “Delivery System and Method for Interstitial Radiation Therapy,” by Terwilliger, et al., filed Mar. 26, 2003 (WORLD-01000US8); U.S. Provisional Application No. 60/469,940 entitled “Delivery System and Method for Interstitial Radiation Therapy Using Seed Strands with Custom End Spacing,” by Terwilliger, et al., filed May 13, 2003; U.S. patent application Ser. No. 10/619,928 entitled “Delivery System and Method for Interstitial Radiation Therapy Using Seed Strands with Custom End Spacing,” by Terwilliger, et al., filed Jul. 15, 2003; U.S. Provisional Patent Application No. 60/360,299, entitled “Delivery System and Method for Interstitial Radiation Therapy Using Seed Elements With Ends Having One of Projections and Indentations,” by Terwilliger et al., filed Feb. 26, 2002 (Attorney Docket No. WORLD-01003US0); U.S. patent application Ser. No. 10/162,547 entitled “Delivery System and Method for Interstitial Radiation Therapy Using Seed Elements with Ends Having One of Projections and Indentations,” by Terwilliger, et al., filed Jun. 4, 2002 (WORLD-01003US1); U.S. Provisional Patent Application No. 60/360,260 entitled “Delivery System and Method for Interstitial Radiation Therapy,” by Terwilliger et al., filed Feb. 26, 2002 (Attorney Docket No. WORLD-01004US0); U.S. patent application Ser. No. 10/132,930 entitled “Improved Delivery System and Method for Interstitial Radiotherapy Using Hollow Seeds,” by Terwilliger et al., filed Apr. 26, 2002 (Attorney Docket No.: WORLD-01004US1); and U.S. Published Patent Application No. 2004/0102671, entitled “Delivery System and Method for Interstitial Radiation Therapy Using Seed Strands Constructed with Preformed Strand Housing”, by Lamoureux and Terwilliger, filed Nov. 10, 2003, and assigned Ser. No. 10/705,133.

As may be recognized by those or ordinary skill in the pertinent art based on the teachings herein, numerous changes and modifications may be made to the above-described and other embodiments of the present invention without departing from the scope of the invention as defined in the appended claims. For example, the at least one outer wall of the apparatus may take the form of any of numerous different structures or configurations that are currently known, or that later become known, for purposes of moving between retracted and expanded positions, and for carrying a plurality of radiation sources in the expanded position. In addition, the apparatus may include any of numerous different devices or structures that are currently known, or that later become known, for supporting the at least one outer wall, and for manipulating or otherwise moving the at least one outer wall between the retracted and expanded positions. Further, the radiation sources may take the form of any of numerous different low dose and/or high dose radiation sources, such as any of numerous different brachytherapy radiation sources, that are currently known, or that later become known. In addition, the different components or features of the apparatus may be made of any of numerous different materials, and may be constructed in accordance with any of numerous different techniques, that are currently known, or that later become known. Still further, the apparatus of the present invention may be used in accordance with any of numerous different methods or treatments that are currently known, or that later become known. Accordingly, this detailed description of preferred embodiments is to be taken in an illustrated, as opposed to a limiting sense.

1. A brachytherapy apparatus for treating tissue forming an internal body cavity, comprising:
at least one outer wall movable between (i) a retracted position spaced inwardly relative to tissue forming the internal body cavity, and (ii) an expanded position spaced outwardly and contiguous to tissue forming the internal body cavity; and
a plurality of radiation sources coupled to the at least one outer wall and movable therewith between the retracted and expanded positions for transmitting radiation in the expanded position into the tissue forming the internal body cavity.

2. An apparatus as defined in claim 1, wherein the apparatus defines an elongated axis, and the at least one outer wall is movable radially relative to the axis between the retracted and expanded positions.

3. An apparatus as defined in claim 1, wherein the plurality of radiation sources are spaced relative to each other, and are at least one of (i) embedded in, (ii) mounted on, and (iii) form, the at least one outer wall of the apparatus.

4. An apparatus as defined in claim 1, wherein the at least one outer wall is defined by a plurality of axially-elongated, radiation-emitting members angularly spaced relative to each other and movable between (i) the retracted position spaced radially inwardly toward the elongated axis, and (ii) the expanded position spaced radially outwardly relative to the elongated axis, and wherein each of a plurality of the radiation-emitting members includes at least one radiation source for transmitting radiation into the adjacent tissue forming the internal body cavity.

5. An apparatus as defined in claim 1, wherein in the expanded position at least one of the at least one outer wall and tissue forming the internal body cavity substantially conformably contacts the other.

6. An apparatus as defined in claim 5, wherein the at least one outer wall is flexible and substantially conforms to the tissue forming the internal body cavity.

7. An apparatus as defined in claim 1, wherein the at least one outer wall is defined by a flexible membrane extending about a periphery of the apparatus.

8. An apparatus as defined in claim 7, wherein the flexible membrane is defined by a balloon.

9. An apparatus as defined in claim 7, further comprising an external flexible membrane, and an internal flexible membrane located radially inwardly relative to the external flexible membrane, wherein the plurality of radiation sources are located between the internal and external membranes.

10. An apparatus as defined in claim 9, further comprising a plurality of axially-elongated, radiation-emitting members that are angularly spaced relative to each other, that include thereon the plurality of radiation sources, and that are movable with the external flexible membrane between the retracted and expanded positions.

11. An apparatus as defined in claim 1, wherein in the expanded position the plurality of radiation sources are substantially uniformly spaced relative to the respective contiguous tissue surface portions forming the internal body cavity.

12. An apparatus as defined in claim 4, wherein each of at least a plurality of the radiation-emitting members includes a plurality of radiation sources axially spaced relative to each other.

13. An apparatus as defined in claim 12, wherein each of at least a plurality of the radiation-emitting members includes a plurality of radiation sources axially spaced relative to each other and a plurality of spacers located therebetween.

14. An apparatus as defined in claim 12, wherein at least one radiation-emitting member is defined by a first elongated half-shall member, and a second elongated half-shell member fixedly secured to the first elongated half-shell member with the at least one radiation source located therebetween.

15. An apparatus as defined in claim 4, wherein a plurality of the radiation-emitting members define a plurality of axially-elongated apertures therebetween.

16. An apparatus as defined in claim 4, wherein a plurality of the radiation-emitting members are interconnected by a plurality of webs extending therebetween.

17. An apparatus as defined in claim 16, wherein a plurality of the webs define a plurality of axially-extending apertures formed between adjacent radiation-emitting members.

18. An apparatus as defined in claim 1, further comprising first and second radiation sources, wherein at least one first radiation source defines a first predetermined radiation intensity, at least one second radiation source defines at least one second predetermined radiation intensity greater than the first predetermined radiation intensity, and the first and second radiation sources are spaced relative to each other at predetermined locations on the at least one outer wall.

19. An apparatus as defined in claim 18, further comprising indicia representing the location of at least one of the first and second radiation sources to facilitate orienting the apparatus within the internal body cavity.

20. An apparatus as defined in claim 1, further comprising a first support member and a second support member, wherein the at least one outer wall is coupled to at least one of the first and second support members, and at least one of the first and second support members is movable relative to the other to, in turn, move the at least one outer wall between the retracted position and the expanded position.

21. An apparatus as defined in claim 20, wherein the first support member is substantially coaxially mounted within the second support member.

22. An apparatus as defined in claim 21, wherein the second support member includes a tubular portion receiving therein the first support member.

23. An apparatus as defined in claim 22, wherein the first support member includes a proximal end and a distal end, and the distal end includes thereon a stop surface that engages the second member to move the at least one outer wall between the retracted position and the expanded position.

24. An apparatus as defined in claim 1, further comprising a stent including the at least one outer wall.

25. An apparatus as defined in claim 1, further comprising a balloon catheter including the at least one outer wall.

26. An apparatus as defined in claim 1, wherein the plurality of radiation sources are formed by at least one of a radioactive wire and a radioactive seed.

27. An apparatus as defined in claim 4, wherein each of a plurality of the radiation-emitting members defines therein a conduit for receiving at least one of a low dose radiation sources and a high dose radiation source.

28. An apparatus as defined in claim 27, wherein each low dose radiation source is selected from the group including a radioactive wire, a radioactive seed, and a flexible strand including therein at least one radioactive seed.

29. An apparatus as defined in claim 4, wherein each of a plurality of the radiation-emitting members is defined by a flexible strand including therein a plurality of radioactive seeds.

30. An apparatus as defined in claim 29, wherein each strand is formed of a material that is biocompatible and biodegradable.

31. An apparatus as defined in claim 1, wherein the internal body cavity is an excised tumor bed.

32. A brachytherapy apparatus for treating tissue forming an internal body cavity, comprising:
first means movable between (i) a retracted position spaced inwardly relative to tissue forming the internal body cavity, and (ii) an expanded position spaced outwardly and contiguous to tissue forming the internal body cavity; and
second means movable with the first means between the retracted and expanded positions for transmitting radiation in the expanded position into the tissue forming the internal body cavity.

33. An apparatus as defined in claim 32, wherein the first means is defined by an outer wall of the apparatus, and the second means is defined by a plurality of radiation sources.

34. An apparatus as defined in 33, wherein the outer wall is defined by one of (i) a stent, (ii) a balloon catheter, (iii) a plurality of radiation-emitting members angularly spaced relative to each other and movable radially between the retracted position and the expanded position, (iv) a plurality of flexible strands each including therein at least one radioactive seed, and (v) a plurality of radiation-emitting wires, each wire forming a respective radiation emitting member.

35. A method for treating tissue forming an internal body cavity, comprising the following steps:
resecting a tumor site to remove at least a portion of a cancerous tumor and, in turn, forming an internal body cavity at the tumor site;
providing a brachytherapy apparatus including at least one outer wall movable between a retracted position and an expanded position, and a plurality of radiation sources movable with the at least one outer wall between the retracted position and the expanded position;
inserting the brachytherapy apparatus with the at least one outer wall in the retracted position into the internal body cavity at the tumor site;
moving the at least one outer wall and the plurality of radiation sources from the retracted position to the expanded position; and
transmitting radiation from the plurality of radiation sources into the tissue forming the internal body cavity at the tumor site.

36. A method as defined in claim 35, further comprising the step of moving the at least one outer wall in the expanded position into contact with the tissue forming the internal body cavity at the tumor site.

37. A method as defined in claim 36, further comprising the step of substantially conforming at least one of the shape of the at least one outer wall in the expanded position and the internal body cavity to the shape of the other.

38. A method as defined in claim 35, further comprising the step of inflating the apparatus with a fluid to move the at least one outer wall from the retracted position to the expanded position.

39. A method as defined in claim 35, further comprising the step of manually moving the at least one outer wall from the retracted position to the expanded position.

40. A method as defined in claim 35, further comprising the step of substantially uniformly spacing each radiation source relative to the contiguous surface portion of tissue forming the internal body cavity.

41. A method as defined in 35, further comprising the step of providing a brachytherapy apparatus including a plurality of low dose radiation sources, and maintaining the apparatus in the expanded position within the internal body cavity for a period of at least five hours.

42. A method as defined in claim 35, further comprising the step of providing a brachytherapy apparatus wherein the at least one outer wall is defined by a plurality of axially-elongated, flexible, tubular members angularly spaced relative to each other, and moving the plurality of tubular members from the retracted position to the expanded position.

43. A method of defined in claim 42, further comprising the steps of (a) connecting at least a plurality of the tubular members in the expanded position to a source of high dose radiation; (b) introducing the source of high dose radiation into the plurality of tubular members; and (c) emitting the high dose radiation into the tissue forming the internal body cavity.

44. A method as defined in claim 43, further comprising the steps of (d) emitting the high dose radiation for a prescribed treatment period; (e) after expiration of the prescribed treatment period disconnecting the plurality of tubular members from the source of high dose radiation; (f) maintaining the brachytherapy apparatus within the internal body cavity throughout a prescribed non-treatment period; and (g) after expiration of the prescribed non-treatment period, repeating at least once steps (a) through (e).

45. A method as defined in claim 35, further comprising the steps of providing a brachytherapy apparatus including at least one radiation source defining a first predetermined radiation intensity, and at least one second radiation source defining at least one second predetermined radiation intensity greater than the first predetermined radiation intensity; and positioning the brachytherapy apparatus within the internal body cavity such that the first radiation source is contiguous to a first tissue region prescribed to receive a relatively lower dose of radiation, and the second radiation source is contiguous to a second tissue region prescribed to receive a relatively higher dose of radiation in comparison to the first tissue region.

46. A method as defined in claim 42, further comprising at least one of pre-loading or intraoperatively loading radioactive sources into a plurality of tubular members.
